(19) United States
(12) Patent Application Publication
(10) Pub. No.: US 2022/0249188 A1
(43) Pub. Date: Aug. 11, 2022

(54) SURGICAL DRAPE HAVING AN EXTENDABLE SLEEVE FOR COVERING A SURGICAL INSTRUMENT AND METHODS OF MAKING AND USING SAME

(71) Applicants: Mark McBride, Coronado, CA (US); Carola Romero, Coronado, CA (US)

(72) Inventors: Mark McBride, Coronado, CA (US); Carola Romero, Coronado, CA (US)

(21) Appl. No.: 17/169,929

(22) Filed: Feb. 8, 2021

(51) Int. Cl. A61B 46/17

(52) U.S. Cl. CPC A61B 46/17

(57) ABSTRACT

A surgical drape assembly, including a surgical drape having an opening located on the surgical drape, an extendable sleeve assembly operatively connected to the surgical drape, wherein the extendable sleeve assembly is operatively connected to the opening, and a surgical instrument, wherein the surgical instrument is covered by the surgical drape and a portion of the surgical instrument is located within the extendable sleeve assembly when the extendable sleeve assembly is in an extended condition.

### FIELD OF THE INVENTION

The present invention is generally related to a surgical drape that includes an extendable sleeve for use during surgical procedures. The surgical drape for use during surgical procedures of the present invention utilizes an extendable sleeve that is attached to the surgical drape so that a surgical instrument such as a positioning arm of a surgical device can be located (and enclosed) within the extendable sleeve. In this manner, the extendable sleeve can provide protection against contamination of the surgical site due to exposure of the surgical site to the surgical instrument. Furthermore, the surgical drape of the present invention also quickly and easily covers (and encloses) the surgical instrument and is capable of being quickly and easily removed from the surgical instrument after the surgical procedure has been completed. Finally, the surgical drape of the present invention allows for the surgical instrument to be used during a surgical procedure even while the surgical instrument is covered (enclosed) by the extendable sleeve.

### BACKGROUND OF THE INVENTION

Prior to the present invention, as set forth in general terms above and more specifically below, it is known, to employ various types of surgical drapes for use during surgical procedures. Furthermore, it is known to use a variety of surgical instruments during a surgical procedure. Currently, in order to reduce the likelihood of a surgical instrument contaminating a surgical site, the surgical instrument is conventionally sterilized and then covered with a conventional surgical drape or other similar type of covering. There are some surgical devices or instruments that cannot be sterilized and for these to be used in the sterile field requires them to be covered with a sterile drape or covering. In these instances, the covering that is used to cover the surgical instrument is simply draped over the surgical instrument and the covering is then taped or otherwise secured to a base or other similar structure of the surgical instrument so that the covering remains in place on the surgical instrument. While this type of surgical instrument covering is able to adequately cover the surgical instrument, this type of surgical instrument covering does not adequately protect the surgical site from possibly being contaminated by the surgical instrument since the covering does not completely enclose the surgical instrument. Furthermore, the covering may adversely affect the ability of the surgical instrument to properly operate during the surgical procedure since there are folds and other undesirable portions of the covering that may get in the way of the surgical instrument. While these and other various surgical drapes for use during surgical procedures may have been generally satisfactory, there is nevertheless a need for a new and improved, surgical drape which has an extendable sleeve that is capable of enclosing a surgical instrument for use during surgical procedures.

It is a purpose of this invention to fulfill these and other needs in the art of surgical drapes for use during surgical procedures in a manner more apparent to the skilled artisan once given the following disclosure.

The preferred surgical drape having an extendable sleeve for use during surgical procedures, according to various embodiments of the present invention, offers the following advantages: ease of use; lightness in weight; durability; ability to cover a surgical instrument: ability to reduce surgical site contamination by exposure of the surgical site to the surgical instrument; ability to easily cover the surgical instrument; ability to easily remove the surgical drape from the surgical instrument; ability to allow the surgical instrument to be used during the surgical procedure; and ability to provide a plurality of extendable sleeves on the surgical drape to cover a plurality of surgical instruments that may be used during the surgical procedure. In fact, in many of the preferred embodiments, these advantages are optimized to an extent that is considerably higher than heretofore achieved in prior, known surgical drapes for use during surgical procedures.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic illustration of a surgical instrument being covered prior to a surgical procedure, according to the prior art;

FIG. 2 is another schematic illustration of the surgical instrument being covered prior to a surgical procedure, according to the prior art;

FIG. 3 is a schematic, front view illustration of a surgical drape having an extendable sleeve for use in enclosing a surgical instrument during surgical procedures, according to one embodiment of the present invention;

FIG. 4 is a schematic, rear view illustration of the surgical drape having an extendable sleeve for use in enclosing a surgical instrument during surgical procedures, according to one embodiment of the present invention;

FIG. 5 is a schematic illustration of the surgical drape having an extendable sleeve prior to being located over the surgical instrument, according to one embodiment of the present invention;

FIG. 6 is a schematic illustration of the surgical drape having an extendable sleeve being located over the surgical instrument, according to one embodiment of the present invention;

FIG. 7 is a schematic illustration of the surgical drape having an extendable sleeve being located over the surgical instrument and the extendable sleeve being opened, according to one embodiment of the present invention;

FIG. 8 is a schematic illustration of the surgical drape having an extendable sleeve being located over the surgical instrument and the extendable sleeve being located over the surgical instrument, according to one embodiment of the present invention;

FIG. 9 is a schematic illustration of the surgical drape having an extendable sleeve being located over the surgical instrument and the extendable sleeve being advanced along a portion of the surgical instrument, according to one embodiment of the present invention;

FIG. 10 is a schematic illustration of the surgical drape having an extendable sleeve being located over the surgical instrument and the surgical instrument being activated so that the surgical instrument will extend, according to one embodiment of the present invention;

FIG. 11 is a first schematic illustration of the surgical drape having an extendable sleeve being located over the surgical instrument and the extendable sleeve being further advanced along a portion of the surgical instrument while the surgical instrument is extended, according to one embodiment of the present invention;

FIG. 12 is second schematic illustration of the surgical drape having an extendable sleeve being located over the surgical instrument and the extendable sleeve being further advanced along a portion of the surgical instrument while the surgical instrument is extended, according to one embodiment of the present invention;

FIG. 13 is a third schematic illustration of the surgical drape having an extendable sleeve being located over the surgical instrument and the extendable sleeve being further advanced along a portion of the surgical instrument while the surgical instrument is extended, according to one embodiment of the present invention;

FIG. 14 is a fourth schematic illustration of the surgical drape having an extendable sleeve being located over the surgical instrument and the extendable sleeve being further advanced along a portion of the surgical instrument while the surgical instrument is extended, according to one embodiment of the present invention;

FIG. 15 is a fifth schematic illustration of the surgical drape having an extendable sleeve being located over the surgical instrument and the extendable sleeve being further advanced along a portion of the surgical instrument while the surgical instrument is extended, according to one embodiment of the present invention;

FIG. 16 is a sixth schematic illustration of the surgical drape having an extendable sleeve being located over the surgical instrument and the extendable sleeve being further advanced along a portion of the surgical instrument while the surgical instrument is extended, according to one embodiment of the present invention; and

FIG. 17 is a seventh schematic illustration of the surgical drape having an extendable sleeve being located over the surgical instrument and the extendable sleeve being completely along the surgical instrument and the surgical instrument is fully extended, according to one embodiment of the present invention.

### DETAILED DESCRIPTION OF THE PREFERRED EMBODIMENTS OF THE INVENTION

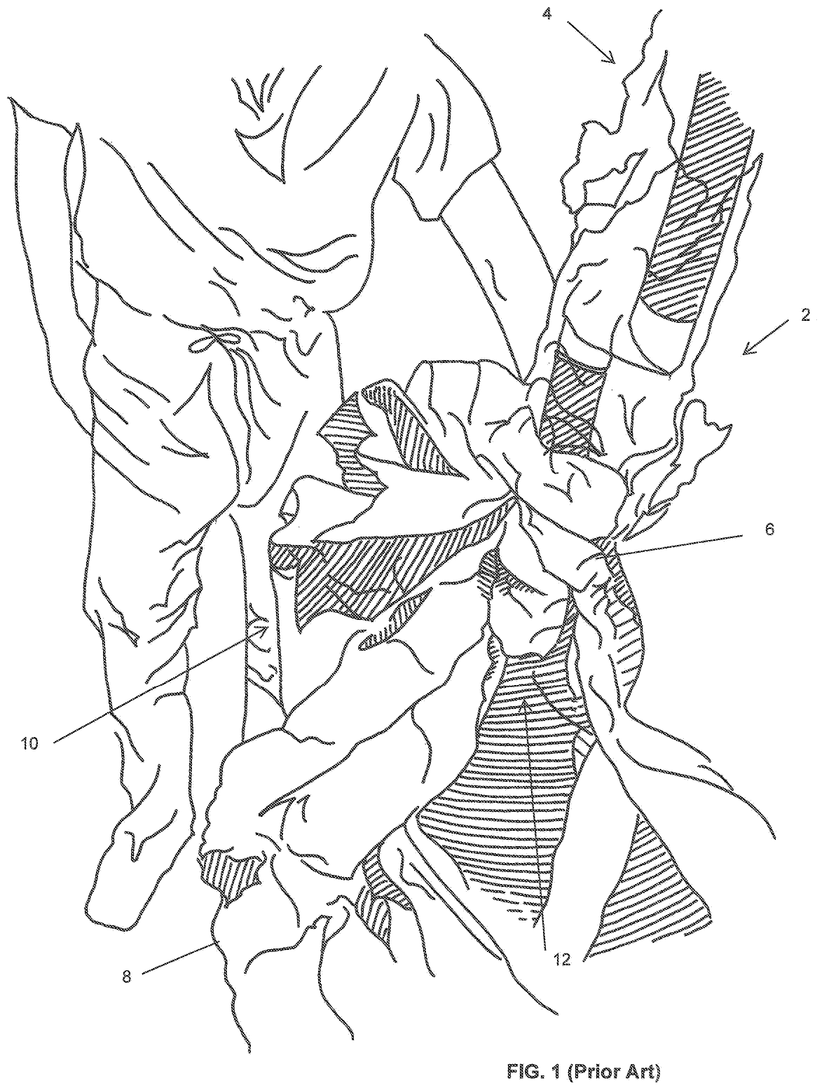

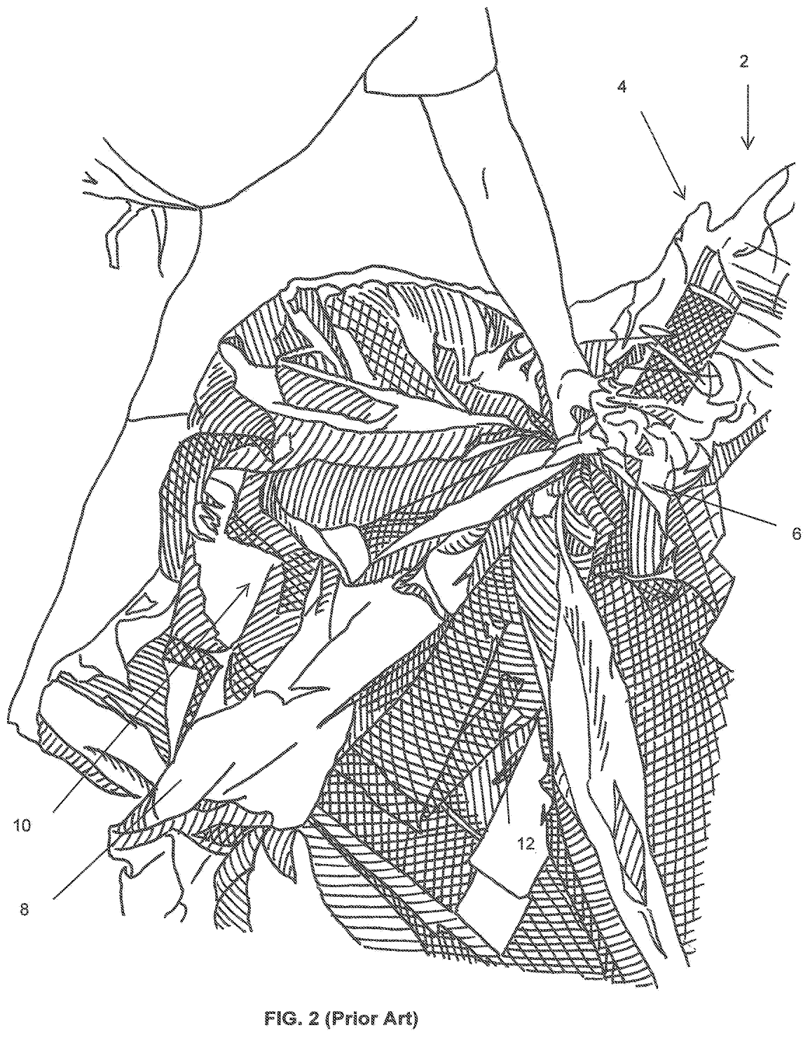

With respect to FIGS. 1 and 2, there is illustrated a well-known draping system 2 for covering a surgical instrument 4 for use during a surgical procedure. As can be seen in FIGS. 1 and 2, draping system 2 includes surgical instrument 4 and surgical drape 8. As discussed earlier, when surgical drape 8 is placed over the surgical instrument 4, the surgical drape 8 is located over the surgical instrument 4 so that the surgical drape 8 covers the surgical instrument 4 including the base 6 of the surgical instrument 4. The operating room personnel then conventionally secure the surgical drape 8 to the surgical instrument 4 and the surgical instrument base 6 using conventional surgical tape (not shown) or other conventional securing devices.

While the surgical instrument 4 is capable of being covered by the surgical drape 8, as can be seen in FIGS. 1 and 2, this known technique also creates undesirable openings 10 and 12 in the drape 8. These openings 10 and 12 can create an opportunity for contaminants located on the surgical instrument 4 that have not been properly removed from the surgical instrument 4 to enter into the surgical site. Furthermore, as can be seen in FIGS. 1 and 2, there are folds and other undesirable portions of the drape 8 that could possible hinder the ability of the surgical instrument 4 to properly operate (or function) during a surgical procedure once the surgical instrument 4 is covered by the drape 8. Finally, after the surgical procedure has been completed, the surgical personnel will then have to spend an inordinate amount of time in removing the surgical drape 8 from the surgical instrument 4.

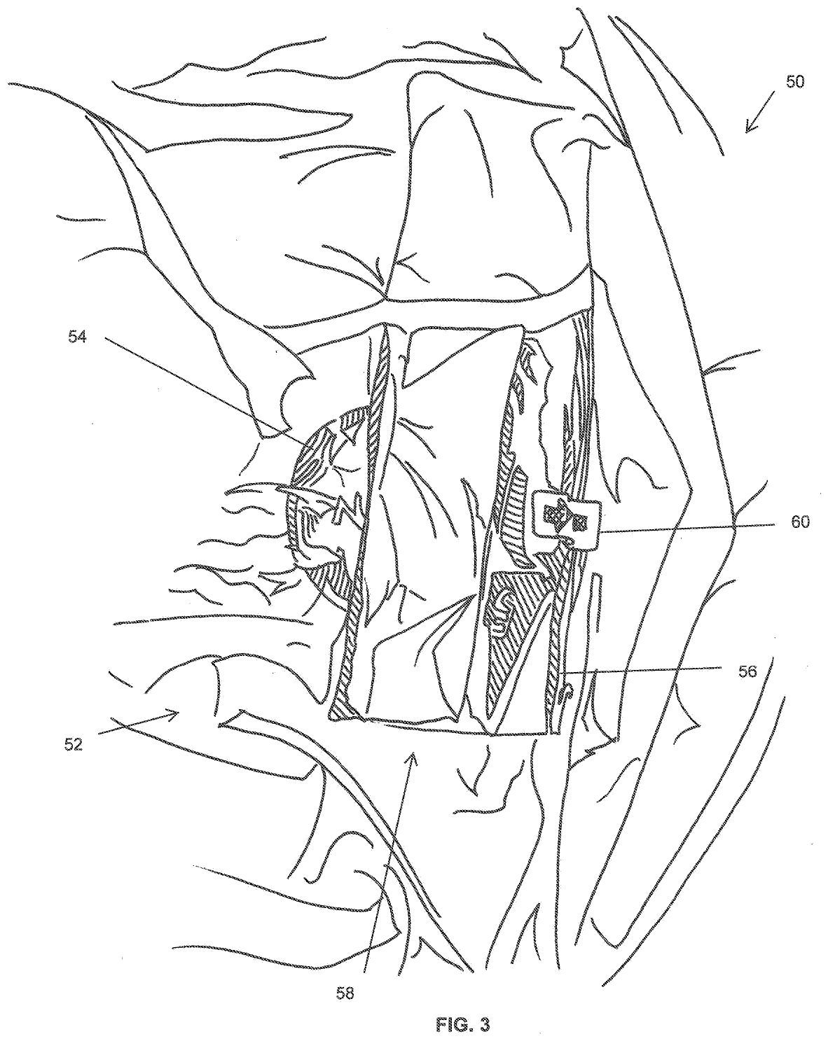

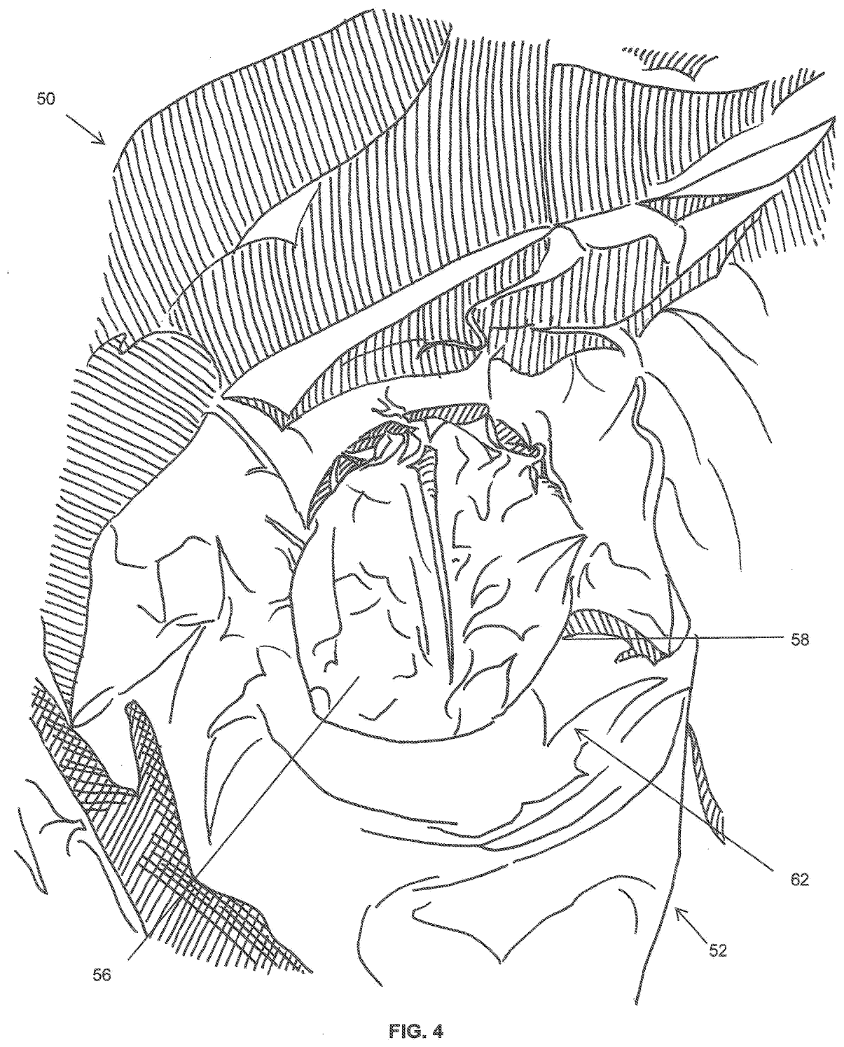

Referring now to FIGS. 3 and 4, there is illustrated a surgical drape 50 having an extendable sleeve 56 for use during surgical procedures. The surgical drape 50 having an extendable sleeve 56 is used to enclose or otherwise cover a surgical instrument during a surgical procedure in order to reduce the likelihood that the surgical instrument will introduce any contaminants into the surgical site while at the same time allowing the surgical instrument to be properly used during the surgical procedure. Furthermore, the surgical drape 50 also quickly and easily covers the surgical instrument and is capable of being quickly and easily removed from the surgical instrument after the surgical procedure has been completed.

With respect to surgical drape 50 having an extendable sleeve 56 for use during surgical procedures, surgical drape 50 includes, in part, conventional surgical drape 52, opening or fenestration 54, extendable sleeve 56, sleeve folds 58, sleeve folds retainer 60, and sleeve/drape attachment 62. Preferably, extendable sleeve 56 is constructed of any suitable clear, durable, high-strength, flexible, medical grade polymeric material such as polyethylene. Also, sleeve folds retainer 60 is constructed of any suitable durable, high-strength, flexible, medical grade adhesive material such as tape that is capable of retaining the sleeve folds 58 in a folded condition until the extendable sleeve 56 is extended, as discussed in greater detail later. Finally, sleeve/drape attachment 62 is any suitable, durable, attachment that is used to secure the end of the extendable sleeve 56 to a periphery around the opening 54 such as heat sealing or any other conventional securing technique.

A unique aspect of the present invention is that the extendable sleeve 56 is to be constructed of a sufficient size and length so to be able to accommodate a variety of conventional surgical instruments particularly once the surgical instrument has been fully extended. Furthermore, the extendable sleeve 56 should be constructed so that the extendable sleeve cannot be pierced or punctured by the surgical instrument or the operating room personnel while the extendable sleeve 56 is being located over the surgical instrument and/or while the surgical instrument is being used during the surgical procedure. Finally, another unique aspect of the present invention is that while only one extendable sleeve 56 is shown, the drape 50 can be constructed to include several extendable sleeves 56 to accommodate other surgical instruments that may be used during a particular surgical procedure.

### Operation of the Surgical Drape

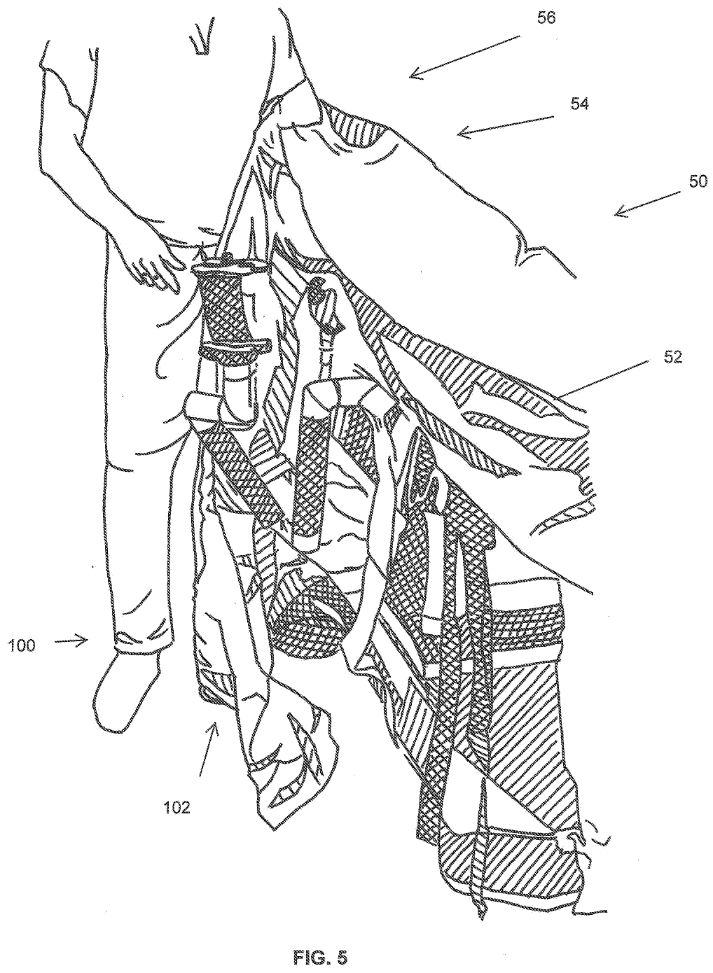

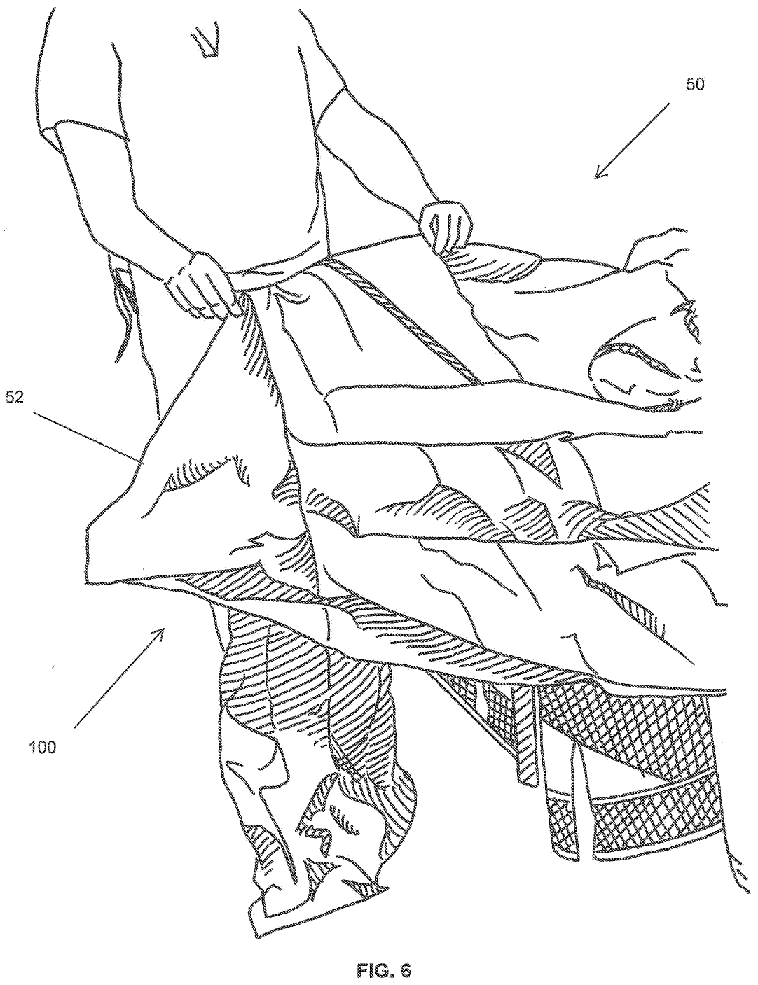

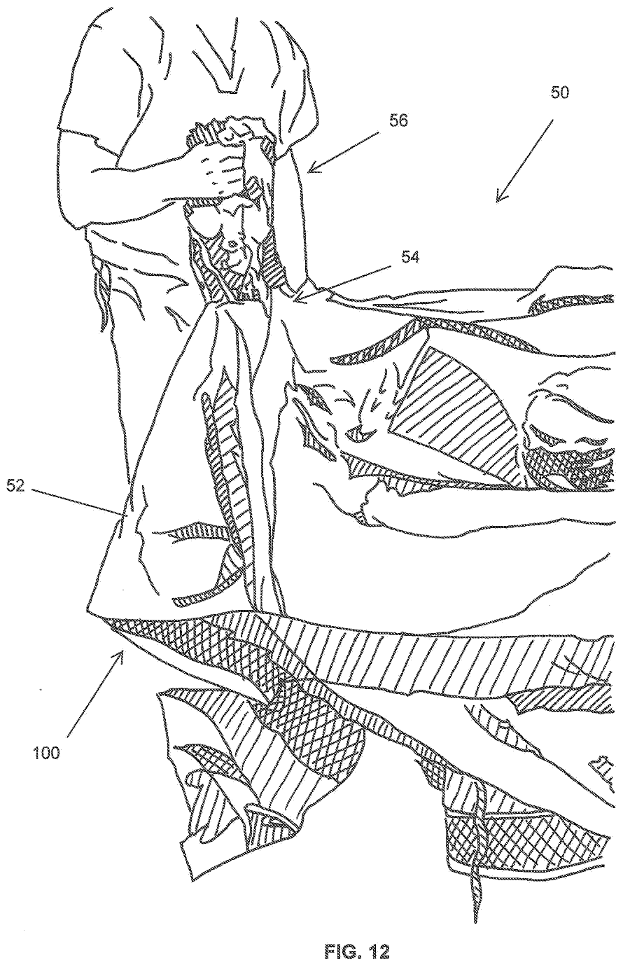

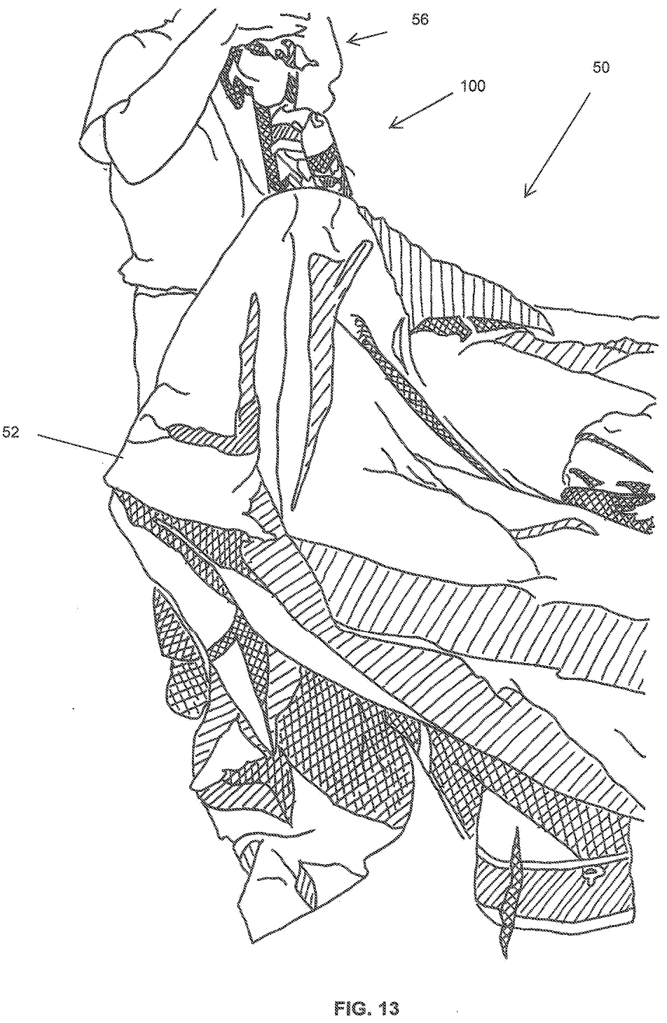

Another unique aspect of the present invention is the use of surgical drape 50 having an extendable sleeve 56 for use during surgical procedures. As shown in FIGS. 5-17, the surgical instrument 100 is conventionally attached to the side of the operating table 102. The surgical drape 50 is located over the surgical instrument 100 (FIG. 5). The surgical drape 50 is then positioned such that the extendable sleeve 58 is located over the surgical instrument 100 (FIG. 6).

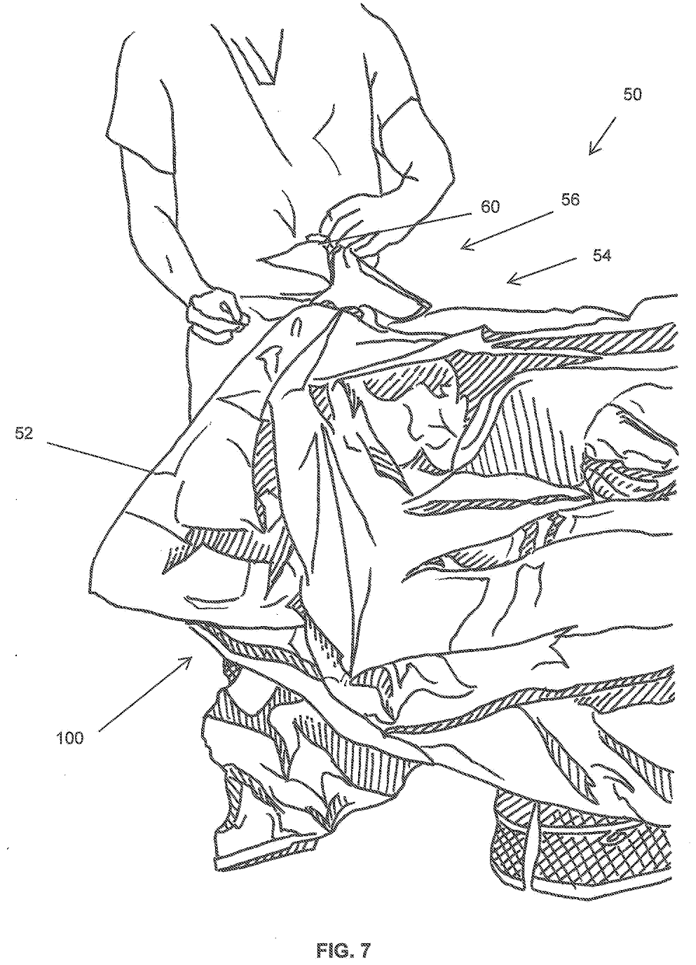

Once the surgical personnel have positioned the extendable sleeve 58 of the surgical drape 50 over the surgical instrument 100, the surgical personnel then cuts or otherwise removes the sleeve folds retainer 60 from the extendable sleeve 58 so that the extendable sleeve 58 can be subsequently extended (FIG. 7).

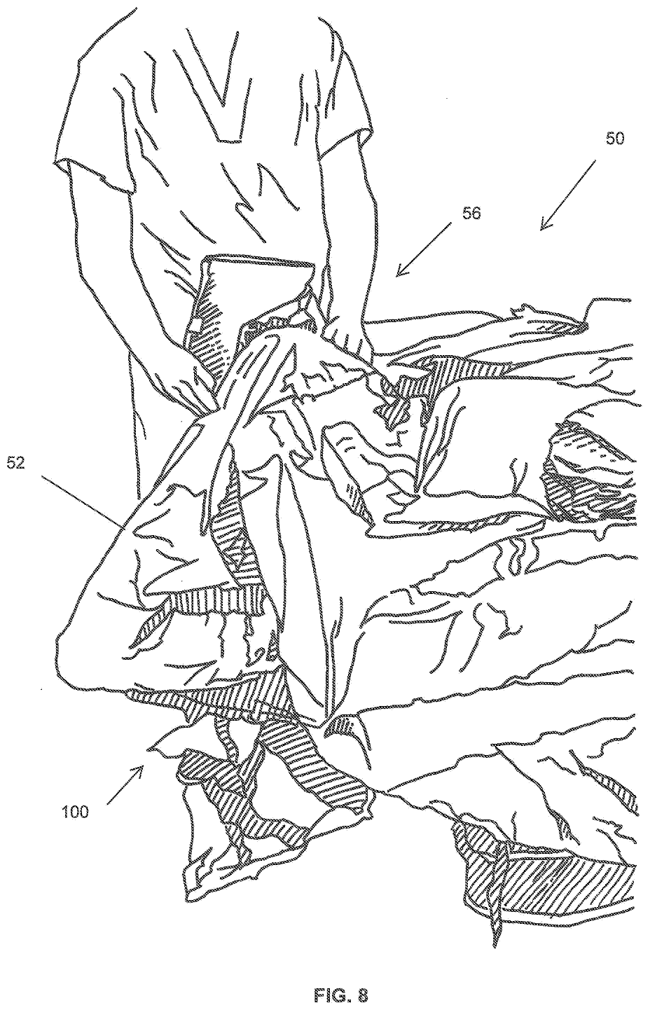

As shown in FIG. 8, the surgical personnel then begins to work or otherwise move the upper portion of the surgical instrument 100 into a portion of the extendable sleeve 58.

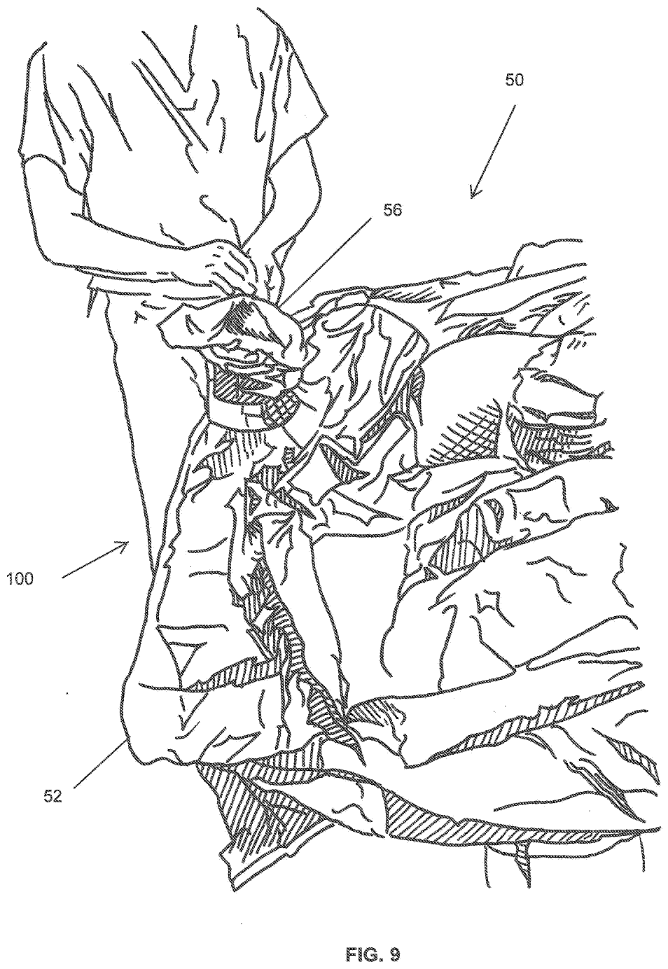

Regarding FIG. 9, the surgical personnel continues to advance the extendable sleeve 58 further down along a length of the surgical instrument 100.

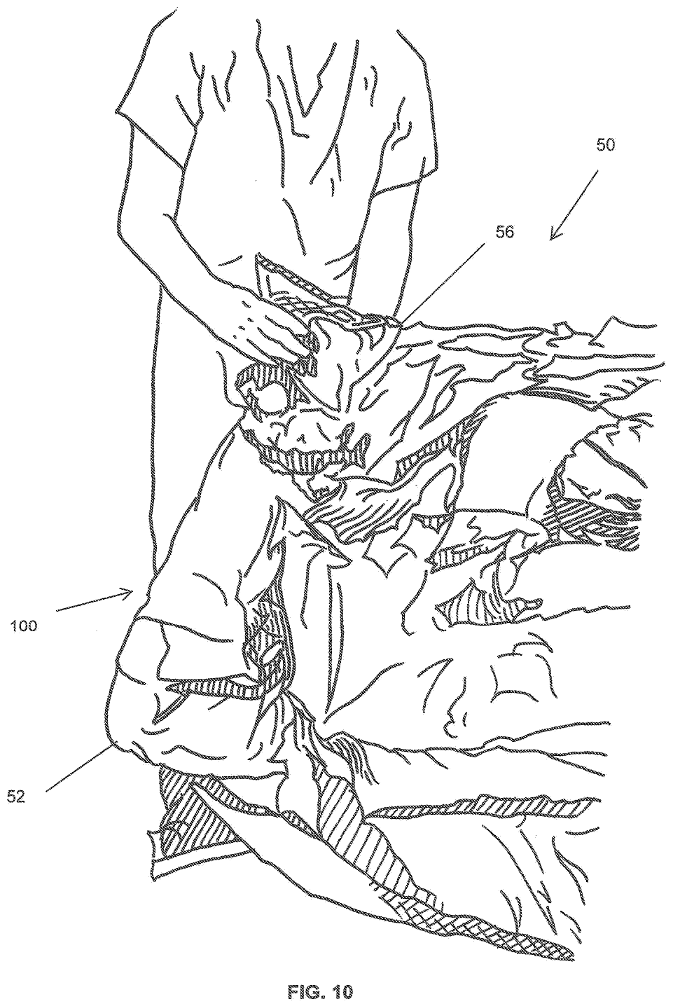

At some point, if the surgical instrument is capable of being extended, the surgical personnel activates the conventional extension mechanism (not shown) of the surgical instrument 100 so that the surgical instrument 100 can be extended (FIG. 10). It is to be understood that surgical drape 50 can be used on surgical instruments that can extend as well as surgical instruments 100 that do not extend or increase in size.

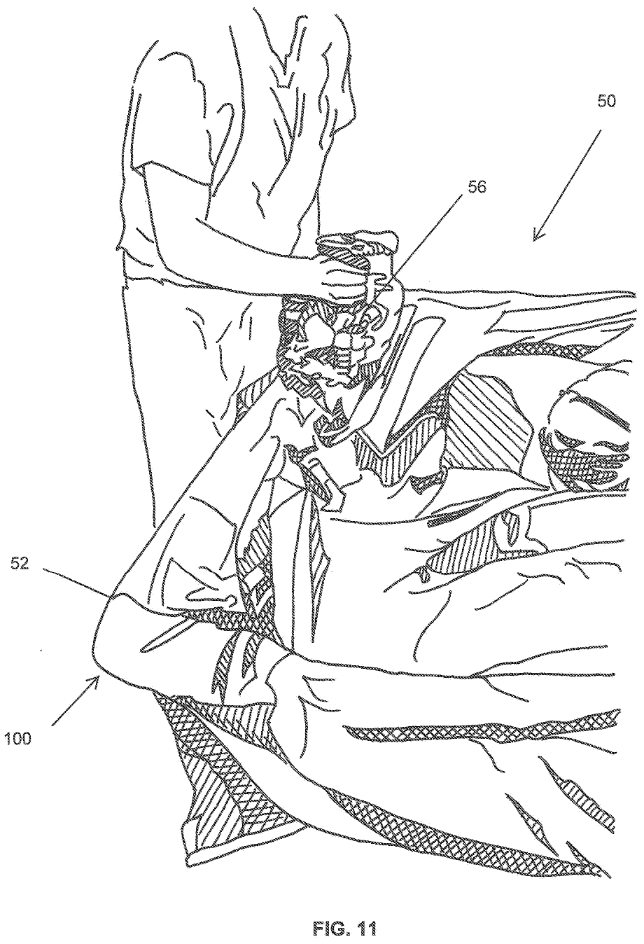

As shown in FIGS. 11-13, the surgical personnel continues to advance the extendable sleeve 58 along the length of the surgical instrument 100.

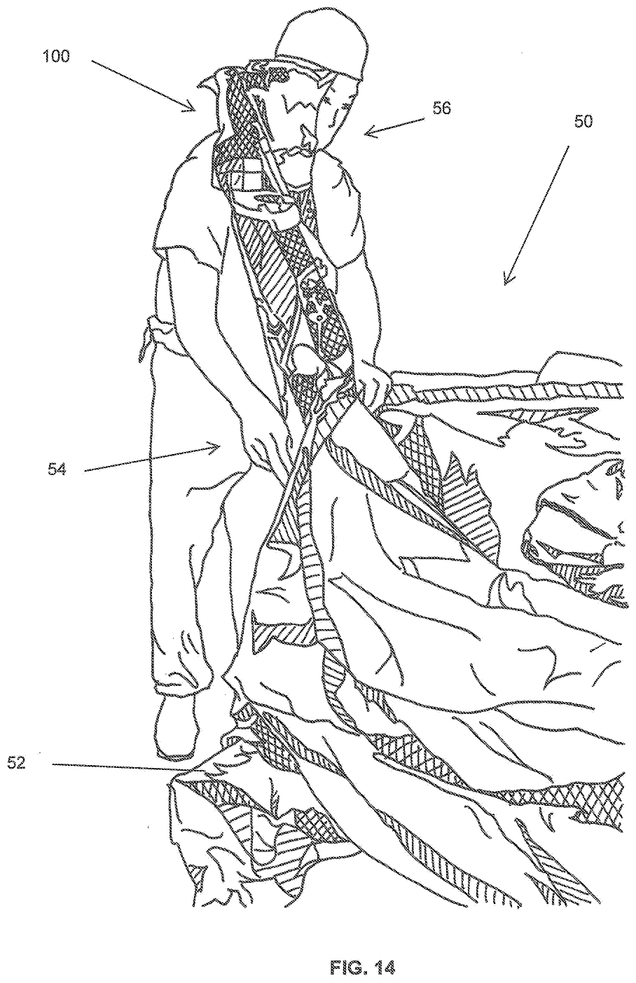

In FIG. 14, the base of the surgical instrument 100 comes into contact with the opening 54 of the surgical drape 50. As this point, the surgical personnel slides the opening 54 and the extendable sleeve 58 over the base of the surgical instrument 100.

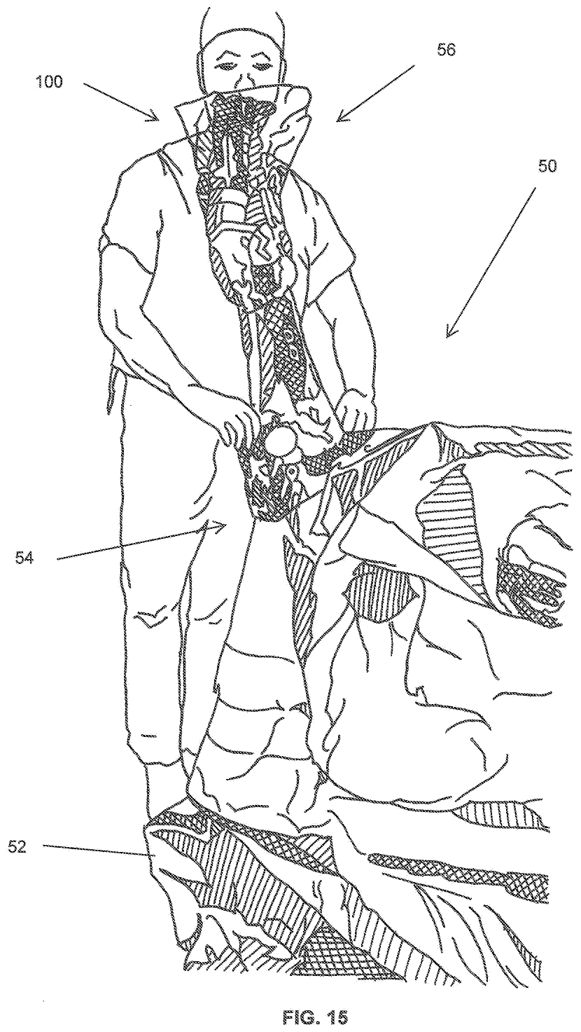

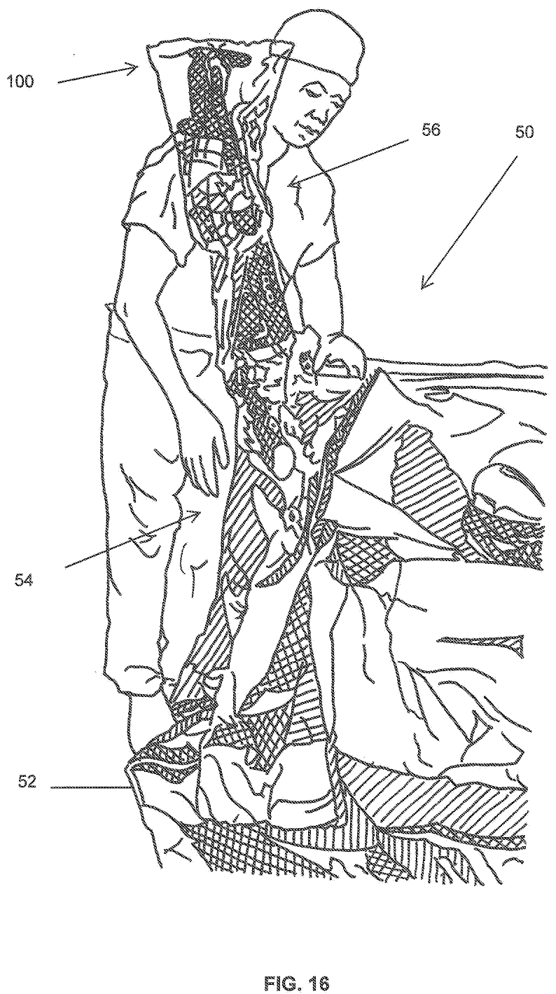

As shown in FIGS. 15 and 16, the surgical personnel continues to advance the opening 54 and the extendable sleeve 58 over the base of the surgical instrument 100. Also, the extendable sleeve 58 is further extended along the length of the surgical instrument 100.

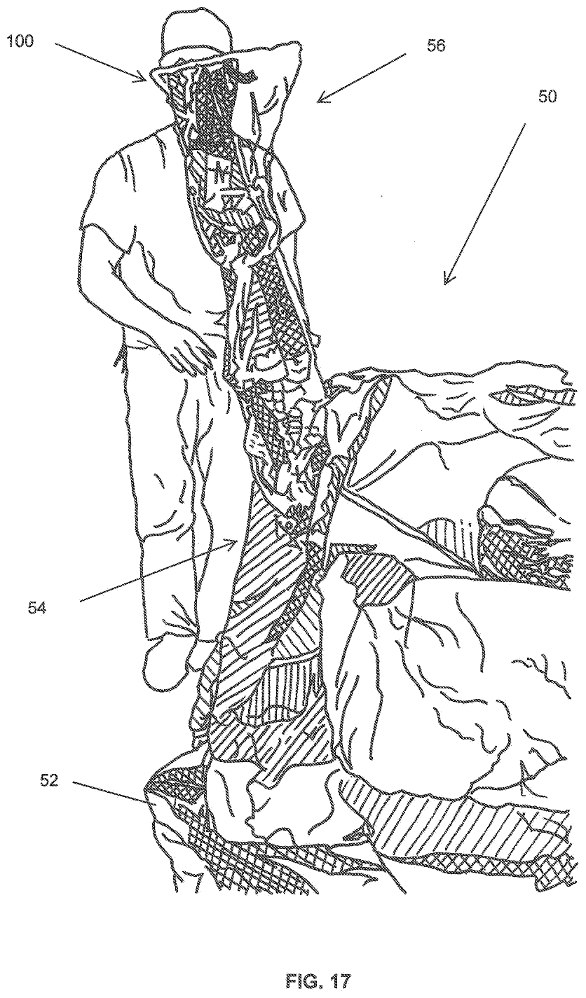

Finally, as shown in FIG. 17, the extendable sleeve 58 is completely extended along the length of the surgical instrument 100. In this manner, the surgical instrument 100 is completely enclosed within the surgical drape 50 and the extendable sleeve 58. As discussed above, another unique aspect of the present invention is that the surgical drape 50 and the extendable sleeve 58 will not adversely affect the ability of the surgical personnel to use the surgical instrument 100 during the surgical procedure.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

It is also to be understood that as used herein and in the appended claims, the singular forms “a” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended and those skilled in the art will recognize, that the invention embraces and is also thereby described in terms of any individual member or subgroup of members of the Markush group.

Therefore, provided herein is a new and improved surgical drape having an extendable sleeve for use during surgical procedures. The preferred surgical drape having an extendable sleeve for use during surgical procedures, according to various embodiments of the present invention, offers the following advantages: ease of use; lightness in weight; durability; ability to cover a surgical instrument; ability to reduce surgical site contamination by exposure of the surgical site to the surgical instrument; ability to easily cover the surgical instrument; ability to easily remove the surgical drape from the surgical instrument; ability to allow the surgical instrument to be used during the surgical procedure; and ability to provide a plurality of extendable sleeves on the surgical drape to cover a plurality of surgical instruments that may be used during the surgical procedure.

In fact, in many of the preferred embodiments, these advantages of ease of use, lightness in weight, durability, ability to cover a surgical instrument, ability to reduce surgical site contamination by exposure of the surgical site to the surgical instrument, ability to easily cover the surgical instrument, ability to easily remove the surgical drape from the surgical instrument, ability to allow the surgical instrument to be used during the surgical procedure, and ability to provide a plurality of extendable sleeves on the surgical drape to cover a plurality of surgical instruments that may be used during the surgical procedure are optimized to an extent that is considerably higher than heretofore achieved in prior, known surgical drapes for use during surgical procedures.

We claim:

1. A surgical drape assembly, comprising;
a surgical drape having an opening located on the surgical drape;
an extendable sleeve assembly operatively connected to the surgical drape, wherein the extendable sleeve assembly is operatively connected to the opening; and
a surgical instrument, wherein the surgical instrument is covered by the surgical drape and a portion of the surgical instrument is located within the extendable sleeve assembly when the extendable sleeve assembly is in an extended condition.

2. The surgical drape assembly, as in claim 1, wherein the extendable sleeve assembly further comprises:
an extendable sleeve having a first end and a second end;
a plurality of sleeve folds located along a length of the extendable sleeve when the extendable sleeve is in a non-extended condition;
a sleeve folds retainer operatively connected to the plurality of sleeve folds when the extendable sleeve is in a non-extended condition; and
a sleeve attachment located on the second end of the extendable sleeve such that the sleeve attachment is operatively connected to the opening.

3. A method of making a surgical drape assembly, comprising the steps of:
providing a surgical drape having an opening located on the surgical drape;
attaching an extendable sleeve assembly to the surgical drape, wherein the extendable sleeve assembly is operatively connected to the opening; and
covering a surgical instrument, wherein the surgical instrument is covered by the surgical drape and a portion of the surgical instrument is located within the extendable sleeve assembly when the extendable sleeve assembly is in an extended condition.

4. The method of making a surgical drape assembly, according to claim 3, wherein the surgical drape assembly further comprises:
providing an extendable sleeve having a first end and a second end;
creating a plurality of sleeve folds along a length of the extendable sleeve in order to create a non-extended condition of the extendable sleeve;
attaching a sleeve folds retainer to the plurality of sleeve folds such that the sleeve folds retainer retains the extendable sleeve in the non-extended condition; and
attaching a sleeve attachment located on the second end of the extendable sleeve to the opening.

5. A method of using a surgical drape assembly, comprising the steps of:
providing a surgical drape having an opening located on the surgical drape;
attaching an extendable sleeve assembly to the surgical drape, wherein the extendable sleeve assembly is operatively connected to the opening and wherein the surgical drape assembly further comprises:
providing an extendable sleeve having a first end and a second end;
creating a plurality of sleeve folds along a length of the extendable sleeve in order to create a non-extended condition of the extendable sleeve;
attaching a sleeve folds retainer to the plurality of sleeve folds such that the sleeve folds retainer retains the extendable sleeve in the non-extended condition; and
attaching a sleeve attachment located on the second end of the extendable sleeve to the opening; and
covering a surgical instrument, wherein the surgical instrument is covered by the surgical drape and a portion of the surgical instrument is located within the extendable sleeve assembly when the extendable sleeve assembly is in an extended condition.

5. method of using a surgical drape assembly, according to claim 5, wherein the method further comprises:
attaching the surgical instrument to a side of an operating table;
locating the surgical drape over the surgical instrument; and
positioning the surgical drape over the surgical instrument such that the extendable sleeve assembly is located over the surgical instrument.

7. The method of using a surgical drape assembly, according to claim 6, wherein the method further comprises:
removing the sleeve folds retainer from the extendable sleeve so that the extendable sleeve can be subsequently extended.

8. The method of using a surgical drape assembly, according to claim 7, wherein the method further comprises:
moving an upper portion of the surgical instrument into a portion of the extendable sleeve located adjacent to the second end of the extendable sleeve.

9. The method of using a surgical drape assembly, according to claim 8, wherein the method further comprises:
moving an upper portion of the surgical instrument along a length of the extendable sleeve.

10. The method of using a surgical drape assembly, according to claim 9, wherein the method further comprises:
activating the surgical instrument so that the surgical instrument can be extended.

11. The method of using a surgical drape assembly, according to claim 10, wherein the method further comprises:
sliding the opening and the second end of the extendable sleeve over a base of the surgical instrument.

12. The method of using a surgical drape assembly, according to claim 11, wherein the method further comprises:
moving an upper portion of the surgical instrument along the length of the extendable sleeve until the expendable sleeve is completely extended and the surgical instrument is completely enclosed with the surgical drape and the extendable sleeve.
